(19) United States
(12) Patent Application Publication
(10) Pub. No.: US 2013/0158394 A1
(43) Pub. Date: Jun. 20, 2013

(54) Devices, Systems and Methods to Detect and Reduce or Prevent Entry of Inflammatory Mediators into Milk Ducts

(71) Applicants: Sylvia Hon, Monterey Park, CA (US); Anahitta Khosraviani, Fairfield, CA (US); Amar Miglani, Palo Alto, CA (US); David Brandon Smith, Patterson, CA (US); Lanping Amy Sung, La Jolla, CA (US); Geert W. Schmid-Schonbein, Del Mar, CA (US)

(72) Inventors: Sylvia Hon, Monterey Park, CA (US); Anahitta Khosraviani, Fairfield, CA (US); Amar Miglani, Palo Alto, CA (US); David Brandon Smith, Patterson, CA (US); Lanping Amy Sung, La Jolla, CA (US); Geert W. Schmid-Schonbein, Del Mar, CA (US)

(73) Assignee: THE REGENTS OF THE UNIVERSITY OF CALIFORNIA, Oakland, CA (US)

(21) Appl. No.: 13/522,324

(22) PCT Filed: Jan. 14, 2011
(86) PCT No.: PCT/US11/21395

§ 371 Date: Mar. 7, 2013

Related U.S. Application Data
(60) Provisional application No. 61/295,003, filed on Jan. 14, 2010.

(51) Int. Cl. A61B 5/00; A61M 31/00; A61F 5/37

(52) U.S. Cl. CPC A61B 5/0091; A61F 5/37; A61M 31/00
USPC 600/431; 128/887; 606/213; 606/228; 604/503

(57) ABSTRACT

Devices, systems and methods are disclosed for measurement and reduction or prevention of fluid movement in lactiferous ducts, detection of diseased conditions in the breasts, and treatments thereof. Such techniques, methods and devices are applicable to a variety of conditions including carcinomas.

This application is a U.S. National Stage Application under 35 U.S.C. 371 of the PCT application with Serial No. PCT/US2011/021395, filed Jan. 14, 2011; which claims priority to U.S. Provisional Patent Application Ser. No. 61/295,003, filed Jan. 14, 2010, the contents of which are hereby incorporated by reference herein in their entirety into this disclosure.

### BACKGROUND OF THE INVENTION

1. Field of the Invention

The present invention relates to inflammation. In particular, the present invention relates to devices, systems and methods to detect and reduce or prevent entry of inflammatory mediators into milk ducts.

2. Background of the Invention

Breast cancer is one of the most widespread and devastating of all cancers. It affects millions of women and men worldwide and claims countless lives yearly. Despite significant progress in this field over the last 50 years, much still remains to be discovered about this sickness, including its very causes, early detection and prevention. One of the many avenues that still need to be explored in depth is methods of prevention before the malignant transformation of the cancer.

As is currently understood, breast cancer is a disease in which the cells within the breast become cancerous and abnormally proliferate. Breast cancer is the second most common and fatal cancer among American women in particular. The chances of developing invasive breast cancer for women are 1 in 8. The greatest threat from breast cancer is that the root cause is unclear and is currently under investigation. Some studies show that breast cancer originates predominantly in epithelial cells lining the lactiferous ducts. With notable exceptions, a high incidence of tumors is in the regions of the lactoferral ducts that are located within a few centimeters of the nipple. But no predictable pattern has been identified for malignancy in any of the approximate 20 lactiferous channels converging at the nipple.

Since the main cause of the disease unknown, it is difficult to develop precise diagnostic tools, effective treatments and therapies. Some studies have shown that high estrogen levels may be a leading cause of breast cancer. However, studies have not succeeded in determining if estrogen directly causes cancer or whether it may simply be a contributing growth factor to the proliferation of cancerous cells.

Studies currently are investigating whether external toxins, such as detergents and free fatty acids, may be directly linked to the development of cells and tumor proliferation. These chemicals are known to cause genetic mutations that can lead to cancer, as well as trigger a chronic inflammatory response, which is a known progenitor of many types of cancer. However, it is unknown if and how these cells become exposed to external toxins. It is widely accepted that fluid can flow out of the nipple, as demonstrated from breast-feeding. Based on anatomical studies, the breast seems to lack any sort of valve system preventing fluid flow into the nipple.

Longstanding evidence suggests that markers of inflammation precede malignant transformation and cancers. In this context it should be noted that the inflammatory cascade is fundamentally a repair mechanism. The “resolution of inflammation” serves to restore injured tissue and the inflammatory cascade during repair utilizes a set of circulating cells (e.g., neutrophils, T-lymphocytes, macrophages), stem cells and several signaling pathways that are also a part of the original embryonic development to restore a tissue to either a scar tissue or in some cases to its original parenchymal function. Therefore elevated levels of inflammatory markers (e.g., cytokines, lymphokines, proteases, growth factors) are an indication that the tissue is in the process of repair.

Therefore, detection of inflammatory markers in lactoferral channels raises the important question of what mechanism has caused injury to the epithelial cells lining along these channels. The answer to this important question opens the opportunity for early detection of inflammation and design of optimal interventions against the inflammation. Most importantly understanding the injury mechanism may suggest ways for prevention of the disease in the first place.

Few studies have identified mechanisms that may cause direct injury to breast tissue and malignant transformation, i.e., damage to the DNA of epithelial cells lining the milk ducts at a level that causes no necrosis or apoptosis. While trauma due to mechanical stresses, heat, radiation or similar injury mechanism can clearly damage soft tissues cells there is little epidemiological support that these forms of injury have a dominating association with the incidence of breast cancer. Therefore there is a question what other mechanisms may exist that could cause damage to milk duct epithelial cells.

There currently exists no technique to prevent breast cancer before malignant transformation. Some relatively crude techniques do exist, including a conventional technique (ductoscopy) to cannulate and/or introduce endoscopes into lactiferous ducts at the time of breast cancer surgery or in the case of “dripping nipples” for the purpose of collection of cancer cells for research, for biopsy and ablation techniques, and for visual inspection and correlation with biopsy histology. However, such technique is unpleasant, very limited and has a number of drawbacks.

Thus, there is a need for new methods of detecting and controlling factors that relate to breast health and inflammation. The methods should be simple to administer, effective and capable of aiding individuals in diminishing or preventing harmful effects of inflammatory mediators relating to breast cancer.

### SUMMARY OF THE INVENTION

The present invention is based on the hypothesis that epithelial cells are harmed by external toxins entering through the duct openings at the surface of the nipple. Thus, there is a need to develop a device that can detect micro liter volumes of fluid movement through potentially open lactiferous channels on the surface of the nipples, and potentially reduce or stop such fluid or contaminant movement.

The present invention is partially based on the hypothesis that inflammatory mediators derived from environmental fluids (e.g., detergents, soap or chlorides present in hot tubs, bathwaters, etc.) enter through open lactiferous ducts on the surface of the nipple. The presence of such injurious fluids into the lactiferous channels and sinuses allows contact with and injury of epithelial cells in the milk ducts and sinuses. Contact with external fluid is preferentially but not exclusively in the vicinity of the nipple close to the entry point of lactiferous channels. The ducts that are subject to fluid entry have an open communication across the epidermis to the outside in the terminal endings at the tip of the nipple. The opening of the ducts may be due to several possibilities: some ducts may have not been covered or they may be opened in the presence of fluids that swell and permeabilize the epidermis covering the nipple (see below). It is an objective of the presenting invention to develop a technique capable of detecting the presence of individual open lactiferous ducts by measuring fluid flow across the nipple and into underlying tissue.

An important issue in this hypothesis is the mechanism that allows fluid entry via the nipples in the duct system. For this it is necessary to study the mechanics of fluid transport in the duct system. In spite of its importance, the mechanics of milk transport in the breast is today still a poorly explored subject. Forward flow out of the ducts is primarily driven by secretion of milk droplets and other fluids from the epithelial cells in the lactiferous alveolae. The fluid secretion into the intraductal compartment generates an elevated fluid pressure at the terminal end of the alveolae and therefore serves as the prime mechanism to raise the fluid pressure in lumen of the alveolae and move milk towards the lactiferous sinuses. Sinuses appear to be milk storage compartment, possibly with increased distensibility compared to the rest of the ducts, so that fluid volume generated in the alveolae can be temporarily stored in them before being discharged via the ampullae through the narrower ducts in the nipple. Due to the relative small dimensions of the ducts in the human breast and the relatively slow generation of fluid by the epithelial cells, the intraductal forward fluid flow is at low Reynolds numbers. But if the breast and the sinuses are externally compressed, the fluid flow through the ducts in the nipple may reach higher Reynolds numbers, to the point of sufficiently high inertial fluid forces to allow generation of fluid jets escaping from the nipple tip. At high or low Reynolds numbers, forward motion of milk out of the ducts requires presence of higher fluid pressures inside the breast than at the nipple where ducts open to the outside. This is the general fluid mechanical requirement during normal milk secretion.

If, however, a duct is not sealed at the nipple and is open to the outside, there is a possibility that external fluids may enter retrograde back into the ducts and sinuses. A requirement for this to take place is generation of a reduced fluid pressure inside the ducts or sinuses relative to the fluid pressure on the outside at the nipple (e.g., atmospheric pressure). How can such a situation be generated?

First it is noted that there is no conclusive evidence for a reversed fluid transport across the epithelial cells via a fluid reabsorption mechanism and consequently a reversed fluid transport across the wall of the ducts back into the interstitial space of the breast. There may be situations, however, when some epithelial cells may stop fluid transport (e.g., during apoptosis), the site becomes leaky for fluid transport into the interstitium so that unimpaired communication between the duct system and the interstitial fluid compartment may be possible. In such a case a reduced interstitial fluid pressure relative to the outside pressure may potentially lead to fluid entry at the tip of the nipple.

Instead, an open duct is at high risk for retrograde fluid entry if the fluid pressure inside a duct is lower then the ambient fluid pressure at its opening at the nipple, thus there exists a fluid pressure drop into the breast. Such a situation can arise readily if a duct is transiently compressed and expanded during any type of breast tissue movement (e.g., by normal respiration, transient breast deformation or deliberate compression). During a duct compression phase, lactiferous fluid is discharged at the tip, and the duct is partially reduced in volume. During the tissue recoil, the duct will elastically recoil to its resting shape (not stay in a compressed configuration) and instead. To achieve this, the duct needs to increase its volume, and in the presence on an incompressible ductal fluid, can do so only by fluid flow backwards from the nipple into the duct. Thus, elastic recoil of compressed and partially discharged ducts is likely to permit fluid entry. If relative small amounts of fluid will enter, due to small compression of the ducts, outside fluid will enter only into those regions of the duct that are in immediate vicinity of the nipple, which may explain the high incidence of ductal inflammation in proximity to the nipple. The less an open duct can be compressed, the closer to the nipple is the environmental fluid that has entered, and the more a duct can be compressed the further fluids can enter into a duct system.

In situations in which all ducts are sealed at the nipple, no retrograde flow into the ducts is possible. Thus, the fundamental problem in inflammation of the breast epithelial cells is the presence of open ducts so that environmental fluids can enter retrograde and initiate an inflammatory reaction on the epithelium. The observation that the ductal system in the vicinity of the nipple is subject to enhanced incidence of lesion formation is in line with the hypothesis of the present invention.

The present invention provides techniques to detect, assess and reduce or prevent introduction of inflammatory mediators that could result in inflammation, diseased states, and possible cancers. Though the disclosed techniques have been largely presented with respect to breast cancer, the diagnostic ideas expressed may be applicable to other similar diseased conditions, including prostate inflammation and cancer, ovarian and colon inflammation and cancer, and certain forms of brain inflammation and cancer. One having ordinary skill in the art would be cognizant of the technique of application of the present disclosure to such other conditions listed above or equivalents thereof after consideration of the present disclosure.

More generally, the present techniques are applicable to all inflammation in and cancers derived from epithelial cells, namely carcinomas, since epithelial cells are potentially exposed to detergents or other agents and carcinomas account for about 85% of all cancers.

In certain aspects of the present invention, techniques are presented for (a) detection of open lactiferous ducts, (b) preventive measures to close them and prevent inflammation and ductal epithelial cancer, and (c) anti-inflammatory/anti-cancer treatment via open channels.

In this disclosure, the methods proposed include use of self-adhesive caps/bandages (“Milk-duct Caps”) that are placed over the nipple/areolar area of the breast and coated for different purposes, including but not limited to:
Milk-duct Caps for the purpose of temporary sealing of transcutaneous lactiferous ducts: Seal with a non-water and -soap soluble lotion/cream lactiferous channels that are open to the outside (transcutaneous) and can serve as potential entry points for environmental toxins;
Milk-duct Caps for diagnostic detection of transcutaneous lactiferous ducts. Milk-duct Caps coated with a liquid (in a sponge-like material) containing fluorescent (or other tracer material) that serve to detect open milk ducts.
Milk-duct Caps for the purpose of sealing transcutaneous lactiferous ducts: Milk-duct Caps coated with a surgical glue to seal transcutaneous lactiferous channels or with cutaneous growth factors/stem cells designed to seal the skin at the point of entry of transcutaneous channels.
Milk-duct Caps for therapeutic purposes: For direct (versus intravenous) administration of anti-inflammatory and anti-cancer drugs into open lactiferous channels. These types of Milk-duct Caps are coated with spongelike material soaked in solutions of anti-inflammatory and anti-cancer drugs; they are worn for selected periods of time until absorption into the milk-ducts by retrograde fluid entry.

There is no comparable technique to reduce or prevent milk duct inflammation, to detect transcutaneous milk ducts, to seal milk ducts, or to treat existing inflammation/epithelial cancers.

### BRIEF DESCRIPTION OF THE DRAWINGS

FIG. 1A shows a side cut view of an exemplary breast when submerged in contaminated aqueous solution.

FIG. 1B shows a side cut view of an exemplary breast when submerged in contaminated aqueous solution and contaminants have entered the milk ducts.

FIG. 1C shows a side cut view of an exemplary breast when submerged in contaminated aqueous solution and contaminants have been prevented from entering milk ducts by a physical barrier.

FIG. 2 shows a side view of a device which may be used to introduce tracers or markers or therapies into the milk ducts of a breast, according to an exemplary embodiment of the present invention.

FIG. 3 shows a schematic view of an imaging system which may be used to sense or detect tracers, markers, contaminants or introduced drugs in the milk ducts of a breast, according to an exemplary embodiment of the present invention.

FIG. 4A shows a top perspective view of an exemplary milk duct cap, according to an exemplary embodiment of the present invention.

FIG. 4B shows a side perspective view of an exemplary milk duct cap, according to an exemplary embodiment of the present invention.

### DETAILED DESCRIPTION OF THE INVENTION

Breast cancer is a prevalent and deadly disease that appears to originate in the epithelial lining of the lactiferous channels. The root cause of breast cancer is still unknown. While hypotheses addressing the biochemical factors of the disease have been explored, there has been little investigation into the possible mechanical causes of the disease. This invention disclosure proposes that toxins from the external environment enter the lactiferous channels via openings on the surface of the nipple and induce mutations to epithelial cells leading to cancer formation. To test this hypothesis a near infrared imaging system has been developed that employs the imaging of Indocyanine Green (ICG), a phosphorescent tracer material. Herein, we highlight our approach for illuminating breast tissue and monitoring the re-emitted fluorescence for detection of open lactiferous channels. Our initial studies include determining the sensitivity and experimental parameters of our imaging system.

Lactiferous channels may not show obvious morphological signs that they are open. Open lactiferous channels that show visible open ducts have been detected in patients with breast cancer and are the subject of studies to collect ductal fluid for the purpose of diagnosis. In a non-symptomatic breast the presence of open ducts will require a more functional test that shows fluid movement inside a duct. This can be achieved by detection of either the entry of a fluorescent tracer (e.g. Indocyanine Green) or escape of fluid (e.g., air) from an open lactiferous duct.

Entry of a tracer into open ducts can be detected by placement at the tracer fluid at the tip of the ducts allowing entry during normal compression/expansion of lactiferous ducts (e.g., due to normal movement of breast tissue while walking breathing etc.) and high resolution imaging of the ducts with infrared fluorescence.

Escape of fluid from an open duct can be detected by external compression of the ducts. One possibility is detection of air (ideal for entry into open ducts as a low viscous medium) escaping from an open and partially fluid-filled lactiferous channel. Such ducts may exist during normal deformation of breast tissue and compression/expansion of the duct so that normal lactiferous fluid is expelled and replaced by air. In this case the nipples are placed under water and the breast compressed while gas bubble formation from the ducts at the tip nipple is recorded.

The incidence of open lactiferous channels in a normal non-symptomatic population at different ages and different menstruating and lactating stages remains to be determined.

Lactiferous channels converge at the nipple. While open to the outside during breast feeding, the majority of ducts appear to be sealed at times not associated with nursing. But the details are uncertain and no measurements exist for the incidence of open ducts in normal individuals, even though open ducts can be detected at the time of cancer diagnosis.

There is no evidence for valves inside lactiferous ducts that could prevent retrograde flow into the breast and of the nature found in several other duct systems in the body (e.g. in veins, lymphatics, the heart). Therefore it appears that the lactiferous channels have to depend on a seal at the nipple in order to prevent return fluid into the breast. The exact molecular mechanisms by which ducts are sealed are largely unexplored. Sealing of a duct may involve several mechanisms:
1. Coverage of duct endings by a keratinocyte layer, the thickness of which is not necessarily uniform and may have in fact some openings.
2. The ducts may be sealed by collapse and closure of the lumen of individual ducts at the nipple, a process that may require epithelial—epithelial attachment within the cells lining the duct. The lumen may also be closed by contraction of myoendothelial cells surrounding the ducts so as to close their lumen, as seen for example in contracting arterioles.
3. Furthermore it is possible that during periods of non-lactation the epithelial cells at the tip of the lactiferous channels may undergo apoptosis so that the channels terminate in the connective tissue of the nipple and may therefore be covered by a double layer of connective tissue and by the keratinocyte layer.

Ducts that fall into the third category are least likely to be open to the outside, while ducts covered by a thin keratinocyte layer are possibly more readily permeabilized if the keratinocyte layer is exposed to external fluids that contain detergents and compromise cell integrity. Ducts that are sealed by a mechanism that relies predominantly on closure of the lumen (case 2) may be readily opened in the presence of detergents, since detergents compromise most cell functions, such as cell-cell adhesion or myocyte contraction. No morphological reconstructions exist in the literature that are of sufficient detail to permit a conclusion about the completeness of the keratinocyte cell layer or connective tissue over the terminal endings of the ducts at the tip of the nipple.

Once fluid has entered into a lactiferous channel it is in direct contact with the epithelium of the breast. The high incidence of epithelial tumors, as compared to tumors that affect other cell systems in the breast, suggests that this contact may be a primary injury mechanism.

Our hypothesis serves to generate new opportunities to deal with the breast cancer disease. Most important is the new opportunity to prevent the disease by screening for the presence of open ducts and minimizing the exposure to environmental fluids that are proinflammatory. Specifically, in the presence of open ducts there exist the following preventive strategies:
Prevention by minimizing/eliminating submergence of nipples in bath water and entry of fluids that causes injury to ductal epithelial cells (e.g. avoid soap baths, hot tubs with detergents, etc.);
Temporary closure of open ducts (by placement of surgical glue into the tip of a duct, use of creams that seal to tip of open ducts and do not dissolved in environmental fluids (detergents etc.), microsurgical closure of skin over open ducts, placement of a seal cap over the nipple that is water tight, growth of keratinocytes/fibroblast/epithelium [derived from existing skin cells or stem cells] over the tip of the lactiferous ducts, etc.) in cases of future pregnancies;
Permanent closure of open ducts (by placement and closure of sutures around the ducts, closure of the skin over an open duct, microsurgical removal of a duct) in cases when no further pregnancy and nursing is planned.

The current hypothesis puts forward a mechanism for generation of inflammation in the breast due to unintended retrograde flow of external fluids into the lactiferous ducts. The retrograde flow is possible in unsealed ducts with open communications to the outside across the skin.

Association with Known Risk Factors for Breast Cancer: Our hypothesis is supported by a range of epidemiological results of the risk factors in breast cancer, including the high incidence of breast cancer in countries with hot tub and bath cultures that include soaps, detergents and other ingredients that can cause injury to epithelial cells. The hypothesis is also supported by the relatively high incidence of tumors in those segments of the ducts/sacs that are located in proximity of the nipple.

There is evidence to suggest that obesity may be a risk factor for breast cancer. Obesity is associated with infiltration of macrophages into adipose tissue as well as activation of matrix metalloproteinases. Such proteolytic activity will cause extracellular receptor cleavage and interstitial protein breakdown and as such may interfere with cell adhesion receptors that are involved in sealing of milk ducts. Furthermore stretch of the skin of the breast by expanding adipose tissue tends to stretch the skin at the nipple and shorten it. In this case the ducts will shorten and more of the duct may loose their seal and allow open communication with the outside.

There may also be an association with estrogen. Estrogen may be anti-apoptotic for cancerous cells (i.e., injured cells that have broken DNA and are precarcinogenic).

There is today strong emphasis on a search for a genetic basis for this disease. But it needs to be recognized that history of this disease is relatively short on a scale that is in line with mutations due to genetic pressure. Furthermore today's geographic incidence patterns of breast cancer suggest that environmental influences may be a dominant risk factor. The current focus is predominantly on environmental issues that may trigger the disease. But there is also a possibility that molecules (e.g. epithelial adhesion molecules) may have genetic polymorphysms that lead to incomplete seals of the ducts, reduced skin thickness and other anatomical features of the nipple that are associated with a increased incidence of open ducts.

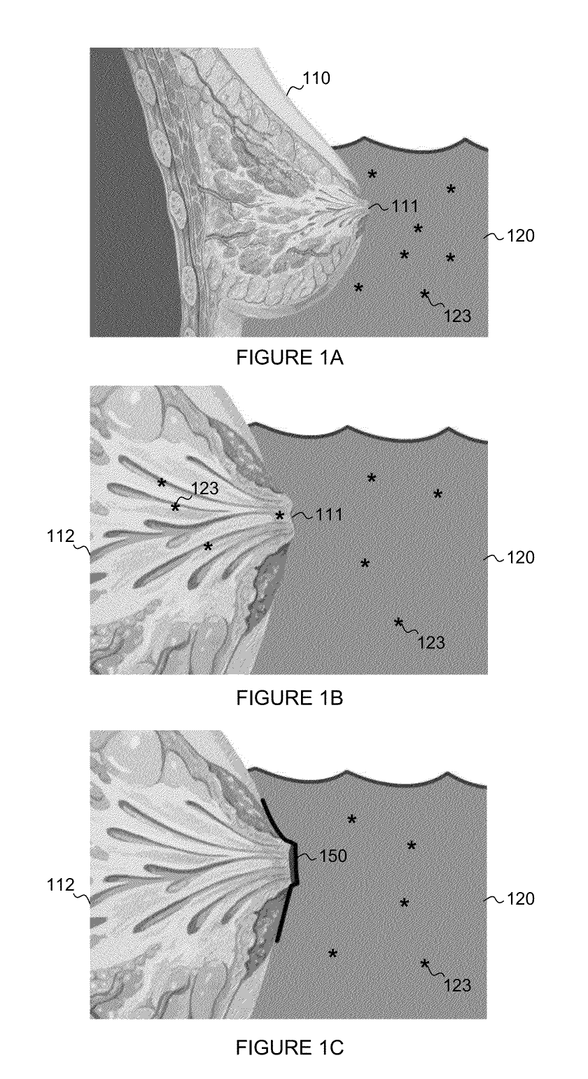

FIGS. 1A, 1B and 1C show an exemplary side cut out view of a breast 110 exposed to an aqueous solution 120 containing one or more contaminants 121. FIG. 1A shows the initial exposure of the breast 110 to the aqueous solution 120 such that the nipple 111 is submerged at least partially or completely within the aqueous solution 120. As described elsewhere the aqueous solution 120 may be, for example, a hot tub, pool, or other water based environment containing one or more contaminants 121. Contaminants 121 may be broadly defined as chemicals, bacteria, viruses, organisms or any other foreign particle which entrance into the body may cause an adverse reaction. As shown in FIG. 1B, certain contaminants 121 may enter into the breast 110 through the nipple 111 and become entrapped or contained within the milk ducts 112. Such process may be exacerbated when the aqueous solution is warm or hot as such temperature may serve to loosen up the nipple 111 to allow more aqueous solution 120 into the milk ducts 112, carrying with it contaminants 121.

As will be described in more detail later, one aspect of the present invention relates to the detection of contaminants 121 which have entered into the milk ducts 112 through the nipple 111. Another aspect of the present invention is the reduction or prevention of the entry of contaminants 121 into the milk ducts 112 by placement of a physical barrier, such as 150, onto the surface of the nipple 111 so as to block the entry point to the milk ducts 112. Other techniques are also possible and within the purview of the present invention after consideration of the present disclosure.

Currently there is no solution or technique to detect the transfer of external toxins such as chlorine through open ducts in the nipple and into breast tissue. It is important to address this problem specifically for early breast cancer detection and prevention. This technique will be used as a diagnostic tool to detect whether a patient is predisposed to developing breast cancer due to an open duct that allows the transfer of external toxins into the breast. In order to detect an open lactiferous channel in the nipple; the objective is to design a technique or device that will measure micro-liter fluid flow through these duct openings. The general design will consist of a technique that is minimally invasive, safe to use on humans, easy to operate, and adaptable for different nipple anatomies.

Research laboratories would be able to use the present technique to study the mechanical properties of the breast in addition to breast cancer. With the use of this technique, research laboratories can verify whether there is a correlation between open lactiferous channels and breast cancer. If a strong correlation is found, then this technique will be intended for women above the age of 25. This diagnostic method would be performed during routine check-up and mammogram screenings. Physicians would administer this diagnostic test to determine whether lactiferous channels are open to the external environment. If exposed, preventative measures may be taken such as temporary sealing of the opened ducts. Overall, this solution would benefit patients with exposed lactiferous channels.

The present invention is based on a new hypothesis about the origin of inflammation and malignant transformation in the lactiferous ducts of the breast and the origin of inflammation and malignant transformation of epithelial cells of the milk ducts, present in many forms of breast cancer. The present subject disclosure proposes to use exemplary methods to screen individuals for possible risk of open lactiferous ducts (before and after malignant transformations), and immediately seal these ducts in order to reduce the risk for fluid entry from the outside. The present diagnosis is designed to provide a possibility for early identification of individuals during screening that are at risk for entry of inflammatory mediators from the environment (e.g., soap and cosmetic, inflammatory antiseptic agents like chlorides, bacterial-, virus-, fungal products, such as in hot tub water, or any other fluid that has an inflammatory effect on epithelial cells and could enter the breast through open milk channels in the nipple). There is currently no method to detect inflammation in the breast at a time before inflammation and early malignant transformation that may lead afterwards to breast cancer.

The present invention includes a number of variations, including one that comprises diagnostic and therapeutic components:
to detect at any age the entry of inflammatory mediators into a normal or diseased breast at the level of the lactiferous ducts in the nipple with a contrast medium detection technique;
to seal (with several different techniques) open lactiferous ducts with a microsurgical, pharmacological, or physical impediment technique and prevent entry of external inflammatory mediators into the lactiferous ducts and generation of epithelial inflammation and malignant transformation;
to collect fluid from open ducts and test for presence of malignant cells and in the presence of early malignant cells in open ducts to inject anti-tumor agents by microinjection and/or filling of ducts by repeated compression and expansion with periodic external tissue compression.

Various techniques for diagnosis are possible. One such non-limiting example of diagnosis of openly communicating lactiferous ducts is described, but as one having ordinary skill in the art would appreciate, many other techniques are also possible and within the scope of the present application.

In one exemplary embodiment, a contrast medium (e.g., indocyanine green, isosulfane blue, or any other contrast medium that can be detected in the breast with imaging techniques) is applied to the tip of the nipple. Its possible entry into openly communicating lactiferous ducts is facilitated by a suction pressure inside the milk ducts generated by mechanical compression and expansion of the breast tissue. The lactiferous ducts at the tip of the nipple and in tissue layers deeper into the breast are then examined with an imaging technique (with resolution sufficient to detect individual ducts). The presence of contrast medium inside the nipple or the underlying breast tissue serves as indicator for open ducts. For example, detection of indocyanine green, approved for i.v. infusion, allows near infrared fluorescent imaging into tissue layers about 1 cm underneath the skin, a distance that is sufficient for detection of openly communicating lactiferous ducts.

The test for entry of contrast medium may be carried out either without (i.e., on dry skin) or with fluid pretreatment of the dermis (wet skin) over the nipple to expose potential leakage sites that are only detected after swelling of the dermis in the presence of water, soap or other inflammatory mediators. For this purpose the nipple will be exposed to a fluid filled microenvironment (e.g., a sealed cup filled with fluid, wet sponge) for a preselected period of time (equivalent to the duration of a typical bath in which the nipple is exposed to bath water). Duct fluid will be collected from open channels and examined by existing techniques for presence of malignant cells or molecular markers in the duct fluid.

If no malignancy is detected open ducts will then be sealed either with a transient technique (before pregnancies) or with a more permanent surgical technique (e.g., after a final pregnancy). These include, but are not limited, to the use of surgical glues injected directly into the open channels at the tip of the nipple, or placement of a surgical suture around the nipple to compress the bungle of lactiferous ducts that converge towards the nipple tip, or other temporary solutions presented in more detail below. The method may also include pharmacological enhancement of cutaneous smooth muscle contraction around the lactiferous ducts, transfection or pharmacological treatment of smooth muscle cells to enhance their contraction, or transfection of epithelial cells to enhance inter-epithelial cell adhesion and lumen closure of the ducts.

If malignancy is detected, the ducts may be filled by cannulation and/or periodic compression and expansion of open ducts with anti-tumor therapy (e.g., anti-tumor cell antibodies, microtububule inhibitors, gene regulators, enzyme inhibitors, DNA/RNA transcription regulators, DNA synthesis inhibitors, DNA Intercalators/Crosslinkers and others). After confirmation that there is no malignancy the channel will be sealed by surgical technique as described above.

An imaging technique for near infrared detection of indocyanine green in the nipple may be used. Further studies to study malignant transformation after entry of inflammatory mediators into milk ducts under experimental conditions may be performed. The exact inflammatory mediator(s) that may cause malignant transformation in milk duct epithelium of man are currently unknown and need to be explored.

The present invention has numerous applications including, but not limited to, methods to develop optical near infrared or radiographic screening techniques for detection of open channels in the breast, e.g., with use of specialized near infrared imaging cameras. The present discovery also allows for new applications of surgical glues or design of specialized sutures for optimal placement in this technique.

The devices and methods according to the present invention should meet certain performance, health and safety, and size and weight requirements. For example, the device should preferably have high sensitivity and be accurate to measure fluid transfer to +0.05 microliters, based on the dimensions of lactiferous channels. It should be compatible with various nipple anatomies, and the testing procedure should not distort the nipple mechanical properties or configuration in a way that leads to erroneous data. The device should be able to discriminate between flow into the nipple, and flow out of the nipple, and its measurements should be accurate and reproducible to within ±5%. In terms of health and safety, no toxic materials should be used, no radioactive tracers should be present as flow may potentially enter the nipple. Furthermore, the scanning device should not use ionizing radiation if possible, as excess ionizing radiation is a known carcinogen. Additionally, nothing that could disturb epithelial tissue and lead to an inflammatory response should be used. Nothing that could agitate the epidermal layer should be used. The size and weight should preferably be such that it is easily handled by a health care worker in a field, with ideal device dimensions preferably being 12″-12″-12″, and maximum weight of device preferably about 10 kg or less.

Various imaging techniques may be used to detect the flow of substances through the milk ducts including, but not limited to, computer axial tomography (CT) scan, near-infrared (NIR) phosphorescent tracers, and electromagnetic flow meters. Although each has its own attributes and drawbacks, the preferable technique used herein involves near includes NIR.

In using NIR, an objective is to detect an open lactiferous duct in the nipple by using a NIR phosphorescent tracer such as Indocyanine Green (ICG) as a tracer (a dye that is FDA approved for human use). ICG would initially be dissolved in a saline solution at the appropriate concentration and placed at the nipple. The breast would then be compressed in order to induce saline flow through the nipple. If there is an open duct, then theoretically the tracer would enter through the channels and into breast. This tracer can be detected quantitatively by using an optical imaging device and would ultimately provide information on whether fluid can flow into the nipple through an open channel.

Indocyanine Green is a dye that functions at near-infrared frequency. Using NIR fluorophores is useful because it functions at a low frequency, which makes tissue transparent. Therefore the ICG tracer can be detected and imaged within the breast tissue. Additionally, using an NIR detector would provide spatial resolution. An actual image allows for the detection of which duct is open in two dimensions. Manipulation of the focusing lens will make possible the quantitative detection of the depth to which the fluid has traveled. A 785 nm light source would directly project towards the nipple. In response, the ICG will absorb the 785 nm light, re-emitting it at 830 nm. There will be a detection apparatus, which will condition the incoming light in multiple stages. A 785 nm notch filter, followed by an 830 nm band pass filter, will selectively screen out the emission light, while passing the phosphorescent signal. Next, a double-convex lens will serve to determine the depth of the focusing plane, as well as “dimming” any out-of-focus light sources. Finally, a charge-coupled device will capture the image in digital format.

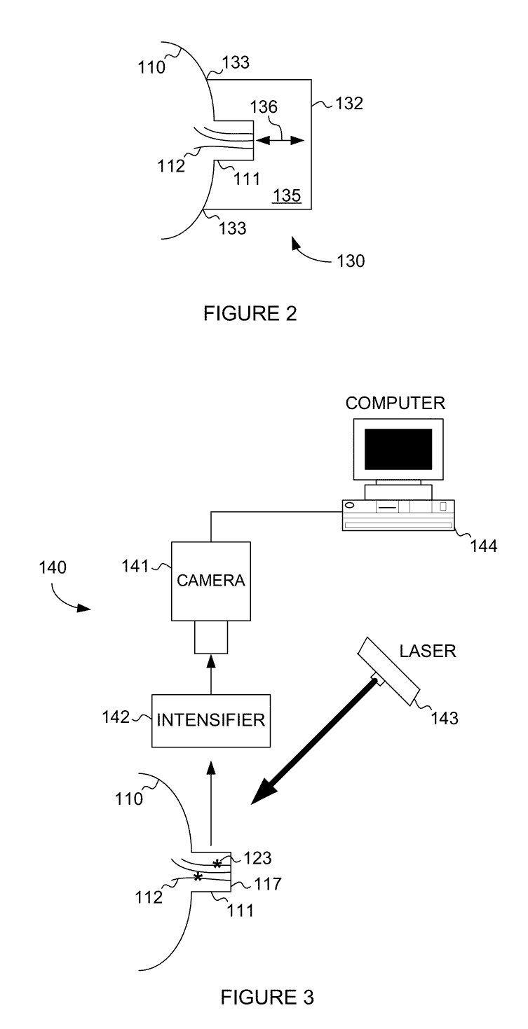

An exemplary system according to the present invention is shown in FIG. 3. This preferred system provides the most direct measurement of open lactiferous channels by detecting fluid that has passed into those channels. This design provides the greatest spatial resolution and allows for the identification of which ducts within a nipple are open. The design fulfills the important goal of not presenting a health hazard to potentially at-risk patients since an ultimate goal is to conduct tests on human breasts for diagnostic and preventative measures. NIR tracer imaging requires ICG to conduct fluorescent contrast imaging. ICG is FDA approved for human testing. FIG. 3 displays a diagram of the NIR tracer imaging method. The design consists of two components: (1) a nipple attachment device (see FIG. 2) that will provide mechanical delivery of saline solution containing ICG, (2) NIR CCD imaging camera and filters system (see FIG. 3). The CCD camera and filters are standard parts that can be purchased but the mechanical delivery device requires fabrication. The mechanical delivery device (FIG. 2) should be able to enclose the surface of the nipple without any leakage and allow addition of ICG into the device without creating an internal pressure that may cause deformation of the breast.

While NIR contrast imaging is still considered an emerging technology, there are examples of NIR phosphorescence imaging currently being used in cancer research today. The present invention may be conducted in a number of different manners for reading NIR. One such non-limiting example is described in Sevick-Muraca, E. M. “Fluorescence-enhanced, near infrared diagnostic imaging with contrast agents.” Current Opinion in Chemical Biology 6.5 (2002):642-650, which is incorporated by reference herein in its entirety into this disclosure. Other methods may also be employed and are within the purview of the present invention.

Indocyanine green (ICG) will serve as the NIR tracer, as it is currently FDA approved for human use. In FDA prescribed quantities, there is no known health risk associated with the use of ICG, with the exception of those patients with an adverse reaction to Iodine-based chemicals. ICG absorbs 785 nm light, and re-emits that light at 830 nm. The chemical has a molar mass of 775 g/mol, is electrically neutral, and small enough to potentially pass from the milk ducts into the extra cellular matrix across epithelial cell tight junctions. If pending experiments indicate that epithelial tight junctions are impermeable to ICG, then ICG will be delivered in a solution containing albumin. This is because ICG binds to human serum albumin with no significant changes to its absorption or excitation profile, allowing for transport into the ECM via preexisting albumin pathways. From the ECM, ICG is absorbed into the lymphatic system, where it has already been demonstrated that the body can filter and dispose of the chemical.

The first step in the design formulation is in the construction of a device that can expose the nipple to the phosphorescent tracer fluid, and allow for 2-way fluid transfer if an open duct is present. Such exemplary device 130 in use is shown in FIG. 2. This device 130 can be a simple open-ended container 132 that is contoured to fit against the breast 110 over the nipple 111, and sealed 133 to be watertight using a temporary tissue adhesive or other known technique. Once sealed, the container 132 is filled with Indocyanine Green tracer fluid 135. The patient is then instructed to apply pressure to the breast 110, inducing compression of the milk ducts 112. Releasing the applied pressure will cause the ducts 112 to re-expand, inducing a pressure gradient between the ducts 112 and the outside environment across the nipple 111 serving to produce liquid motion 136. If an open lactiferous channel 112 is present, this gradient will cause a small quantity of tracer fluid 135 to travel into the channel 112.

The next step in the design process is to construct the necessary imaging apparatus, as shown in FIG. 3 as system 140. For this, we will use a near infrared CCD camera 141, sensitive to 830 nm light. Under appropriate magnification from a macro lens, this camera 141 must be able to resolve objects with a cross-section of 50×50 um, while the total image has a cross section of a few centimeters. This resolution will allow for the identification of individual ducts 112 when ICG is present, scanning the entire nipple 111. If this magnification has a negative impact on the total light collected such that the camera 141 is not sensitive enough to distinguish ICG emissions, an NIR light intensifier 142 will be placed between the patient 110 and the camera 141. A 785 nm laser diode in conjunction with a plano-convex lens will serve to generate the necessary source light and illuminate the tissue. In order to ensure that the CCD camera 141 records only ICG phosphorescent emissions, an 830 nm optical band pass filter may be placed in series with the camera and lens. If necessary, a 785 nm holographic notch rejection filter can also serve to further attenuate the source light 143, which likely will be present at high intensity. The CCD camera 141 will then capture an image, which is sent to a computer 144 where it will be stored for analysis.

There is a high probability that residual Indocyanine green tracer fluid will be present in high concentrations on the surface 117 of the nipple 111, due to the delivery method. If this is the case, high intensity emissions originating from this coating will prevent the detection of ICG within the LF channels. In order to remedy that situation, a modification of the design may be implemented which will use frequency domain analysis of captured images to resolve the depth of the emission light. This modification will require a gated intensifier, with a minimum shutter speed of no more than 5 ns duration. Furthermore, a digital-analog converter and various frequency generators and control mechanisms will be necessary to synchronize the laser diode emissions, CCD image acquisition, and computer image processing.

Various embodiments can be used to change the configuration of the detection system. For example, the original prototype consisted of a 70 mW laser diode aligned towards a plano convex lens which was in turn aligned towards the Indocyanine Green (ICG) solution. In addition a band pass filter was placed directly in front of the camera lens to filter all wavelengths of light except for 830 nm. Several modifications were made to the design. First, the placement of the band pass filter was changed from being positioned in front of the lens to being positioned inside the camera lens. The plano convex lens was completely removed from the design. The rationale of its removal is that the laser diode distributed light over a 5 mm×5 mm field of view; therefore the usage of the convex lens is unnecessary. The laser diode which originally ran at 70 mW was adjusted down to 5% of its original power by rearranging the original circuitry. The power supply of the laser was adjusted because as demonstrated from the experiments, the laser provided undesirable background light that could not be filtered. It would generate a false positive result if ignored.

As discussed above, the present invention provides for a number of diagnostic methods using breast duct passage. Now, a more detailed discussion will be presented for methods of reducing or preventing such breast duct inflammation and associated anomalies. Conventional prevention methods include Ductoscopy to cannulate and/or introduce endoscopes into lactiferous ducts at the time of breast cancer surgery or in the case of “dripping nipples” for the purpose of collection of cancer cells for research, for biopsy and ablation techniques, and for visual inspection and correlation with biopsy histology. Also, the “HALO Test” serves to collect discharged fluid from the breast. This technology is different from the present approach and depends on fluid collection, as compared to the present approach which relies on detection of open lactiferous channels by imaging. The “HALO Test” is a diagnostic but not therapeutic approach.

The approach of the present invention is based on the use of Milk-duct

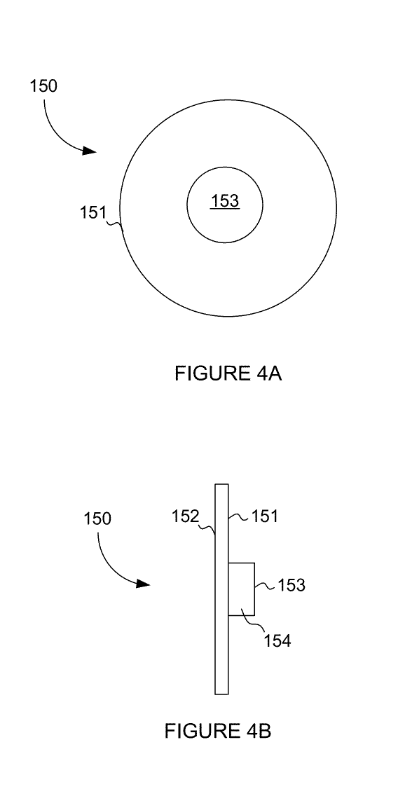

Caps coated with different materials, one for each application. TAs shown in the examples presented in FIGS. 4A and 4B, a Milk-duct Cap 150 can include two concentric circles when viewed from a top perspective. The outer circle 151 comprises an internal surface 152 having a self-adhesive flexible material which attaches directly to the skin (ring shaped) with an interior thimble-shaped 153 with a height and diameter and corresponding volume 154 according to the individual size of the nipple (according to standard sizes to be determined, e.g., small, medium, large size) and designed to avoid any compression of the nipple.

Milk-duct Caps cover the part of the breast skin around the nipple including the areolar region. The adhesive part of the Cap is outside the nipple. The nipple region of the cap is coated/padded with different materials depending on the use (see figure). More specifically:
1. Milk-duct Caps for temporary sealing of transcutaneous lactiferous ducts: (non-soluble coating on the cap to seal) The interior of the tip of the caps over the nipple is layer-coated with non-water-soluble lotions that are not dissolved in environmental detergents/sterilizing agents (e.g., soap, detergents, chlorides) and provide a temporary seal for transcutaneous ducts. The seal materials include hypoallergenic pastes, like zinc oxide (Desitin), and/or equivalent materials. These caps are placed on the nipple preferably before exposure (e.g., soap water in a hot tub, chlorinated water in a swimming pool).
2. Milk-duct Caps for diagnostic detection of transcutaneous lactiferous ducts: (diagnostic coatings on the cap) The interior of the tip of the caps (over the nipple) is layer-coated with a water-absorbent sponge soaked with contrast medium. Selections of possible contrast media are described above. It is worn for a period of time (minutes to hours) before imaging of the nipple/tip of the breast (or any other detection mode of open ducts) to detect transcutaneous lactiferous ducts (as described above).
3. Milk-duct Caps designed for sealing transcutaneous lactiferous ducts: (growth factor coatings on the cap) The interior of the tip of the caps (over the nipple) is layer-coated with a dermal/fibroblast growth factor (e.g., fibroblast growth factor, epi-dermal growth factor, heparin-binding EGF-like growth factor) for cells in the skin or the connective tissue to achieve closure of open transcutaneous lactiferous ducts.
4. Milk-duct Caps for therapy of inflamed or malignant epithelium in the milk ducts: (anti-inflammatory coating on the cap) The interior of the tip of the caps (over the nipple) is layer-coated with an agent that attenuates inflammatory reactions on epithelial cells or serves as anti-tumor treatment in the presence of premalignant or malignant cells (e.g. Tamoxifen Evista [raloxifene hydrochloride] as selective estrogen receptor modulator).

There are many commercial uses of Milk-duct Caps for a variety of home-use, diagnostic, and therapeutic applications including, but not limited to:
1. detection of open transcutaneous lactiferous channels during screening of healthy individuals with potential exposure to pro-inflammatory fluids before detection of lesions/tumors in the breast;
2. temporary closure of transcutaneous channels in individuals (for home use) at risk for fluid entry into the milk-ducts due to temporary exposure (e.g., soap baths, chlorinated swimming pools) at a time when surgical closure of the ducts is contraindicated (e.g. due to future pregnancies, personal choice);
3. regeneration of connective tissue and the dermal layer at the tip of the open transcutaneous channels;
4. direct treatment of early lesions via therapeutic agents through existing open lesions.
   Applications 1, 2 and 3 are predominately for prevention of breast inflammation/cancer. Application 4 is predominately for potential (supplemental) treatment of early lesions/ductal epithelial tumors.

The following references, some whose findings or techniques are discussed or cited above, are hereby incorporated by reference herein in their entirety into this disclosure:

Bilimoria, M. M. (1999). Estrogen replacement therapy and breast cancer: analysis of age of onset and tumor characteristics. Annals of surgical oncology, 6(2), 200-207.

Byrne, C. (1995). Mammographic Features and Breast Cancer Risk: Effects with Time, Age, and Menopause Satus. Journal of the National Cancer Institute, 87(21), 1622-1629.

Higgins, Susan A. “Patterns of reduced nipple aspirate fluid production and ductal lavage cellularity in women at high risk for breast cancer” Breast Cancer Research 2005, 7:R1017-R1022, this article is online at: http://breast-cancer-research.com/content/7/6/R1017

Houston, J. P. “Quality analysis of in vivo near-infrared fluorescence and conventional gamma images acquired using a dual-labeled tumor-targeting probe.” Journal of biomedical optics 10.5 (2005):054010-.

“Indocyanine Green for Injection, USP.” (2006)

Kolin, A. “An electromagnetic catheter-flowmeter.” Circ. Res. 21 (1967):889-900.

Kovar, J. L. “Characterization and Performance of a Near-Infrared 2-Deoxyglucose Optical Imaging Agent for Mouse Cancer Models” Analytical Biochemistry, Vol. 384 (2009) 254-262.

Love, S. M. (2004). Anatomy of the Nipple and Breast Ducts Revisited. Cancer, 101(9), 1947-1957.

Proctor, Kerry A. S. “Cytologic features of nipple aspirate fluid using an automated non-invasive collection device: a prospective observational study” BMC Women's Health 2005, 5:10

“Regulation of Medical Devices: Background Information for International Affairs.” Apr. 14, 1999.

Reynolds, J. S. “Imaging of spontaneous canine mammary tumors using fluorescent contrast agents.” Photochemistry and Photobiology 70.1 (1999):87-94.

Sevick-Muraca, E. M. “Imaging of lymph flow in breast cancer patients after microdose administration of a near-infrared fluorophore: feasibility study.” Radiology 246.3 (2008):734-741.

Sevick-Muraca, E. M. “Fluorescence-enhanced, near infrared diagnostic imaging with contrast agents.” Current opinion in chemical biology 6.5 (2002):642-650.

Sowa, M. G. “Near infrared spectroscopic assessment of hemodynamic changes in the early postburn period” Elsevier Science 27 (2001):241-249.

Suetens, Paul. Fundamentals of Medical Imaging. Cambridge: Cambridge University Press, 2002.

Wood, C. “Re: Conjugated equine estrogen and risk of benign proliferative breast disease: a randomized controlled trial.” Journal of the National Cancer Institute 100.18 (2008):1335; authorreply 1335-6.

Yambe, T. “Recording vagal nerve activity for the control of an artificial heart system.” ASAIO journal 49.6 (2003):698-700

“Ethical principles and guidelines for experiments on animals. Swiss Academy of Medical Sciences. Swiss Academy of Sciences.” Experientia 52.1 (1996):1-3.

Indocyanine Green; MSDS No. SLI2065; Mallinckrodt Baker: Phillipsburg, N.J., Nov. 12, 2003. http://www.sciencelab.com/xMSDS-Indocyanine_green-9924361. (accessed Jan. 19, 2009).

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A method for detecting assessing and promoting breast health through an open lactiferous duct, the method comprising:
exposing a nipple to a fluid containing a contrast medium;
allowing passage of an elapsed period of time; and
detecting the presence of the contrast medium in the lactiferous duct in order to determine if the lactiferous duct is open.

2. The method of claim 1, wherein if the lactiferous duct is determined to be open, further comprising:
collecting a sample of fluid from the lactiferous duct.

3. The method of claim 2, further comprising:
assessing the presence of tumor cells within the collected sample of fluid.

4. The method of claim 3, wherein if tumor cells are detected, further comprising:
introducing anti-tumor compounds into the lactiferous duct.

5. The method of claim 3, wherein if tumor cells are not detected, further comprising:
preventing further fluid flow into the lactiferous duct.

6. The method of claim 5, the preventing fluid flow step comprises:
sealing the lactiferous duct with glue.

7. The method of claim 5, the preventing fluid flow step comprises:
sealing the lactiferous duct via suture.

8. The method of claim 5, the preventing fluid flow step comprises:
sealing the lactiferous duct via a cap.

9. A milk duct cap comprising:
a concave thimble-like inner portion adapted to position over a nipple of a breast; and
an outer rim extending from the concave inner portion;
wherein the outer rim portion contains an adhesive to adhere to an areola of the breast.

10. The milk duct cap of claim 9, wherein an interior portion of the inner portion contains a non-soluble sealant coating which comes into contact with the nipple.

11. The milk duct cap of claim 9, wherein an interior portion of the inner portion contains a diagnostic coating which comes into contact with the nipple.

12. The milk duct cap of claim 9, wherein an interior portion of the inner portion contains a growth factor coating which comes into contact with the nipple.

13. The milk duct cap of claim 9, wherein an interior portion of the inner portion contains an anti-inflammatory coating which comes into contact with the nipple.

14. A system for introducing a therapeutic into a nipple of a breast and then detecting the presence of the therapeutic, the system comprising:
a container having a rim, wherein the rim fits snugly around a nipple and areolar portion of a breast; the container further including a liquid therapeutic within its interior when the container is attached to the areolar portion such that the liquid therapeutic comes into direct contact with the nipple to allow the liquid therapeutic to enter the nipple through milk ducts; and
an imaging system to detect the presence of the liquid therapeutic within the nipple and measure its concentration.

15. The system of claim 14, wherein the liquid therapeutic is coupled with a marker.

16. The system of claim 15, wherein the marker is indocyanine green.

17. The system of claim 14, wherein the rim of the container forms a seal when in contact with the breast.

18. The system of claim 17, wherein the seal is maintained with a temporary body adhesive.

19. The system of claim 14, wherein the imaging system components which can detect near infrared images.

20. The system of claim 14, wherein the imaging system components which can detect indocyanine green.
